(19) United States
(12) Patent Application Publication
(10) Pub. No.: US 2022/0280391 A1
(43) Pub. Date: Sep. 8, 2022

(54) MEDICATION DISPENSING MACHINE AND SYSTEM

(71) Applicant: AINNOTECH A.N LTD, Even Yehuda (IL)

(72) Inventors: Alon Levin, Savyon (IL); Niv Levin, Even Yehuda (IL)

(73) Assignee: AINNOTECH A.N LTD, Even Yehuda (IL)

(21) Appl. No.: 17/636,022

(22) PCT Filed: Aug. 20, 2020
(86) PCT No.: PCT/IL2020/050914

§ 371 Date: Feb. 16, 2022

(30) Foreign Application Priority Data
Aug. 20, 2019 (IL) 268796

(51) Int. Cl. A61J 7/04; G16H 20/13; G16H 40/67

(52) U.S. Cl. CPC A61J 7/0481; A61J 7/0427; G16H 20/13; G16H 40/67; A61J 2200/30

(57) ABSTRACT

A medication dispensing machine for a public domain comprises storage apparatus within which is stored a sufficient supply of each medication needed by all patients of the public domain during a predetermined time period; identification equipment by which each of a plurality of patients of the public domain is able to be authenticated; a controller for coordinating various machine operations; and dispensing apparatus cooperating with said controller and said storage apparatus which is configured to provide a given patient with a prescribed dosage for each of a prescribed combination of medications at a prescribed time following authentication.

### FIELD OF THE INVENTION

The present invention relates to the field of dispensing machines. More particularly, the invention relates to a medication dispensing machine and system.

### BACKGROUND OF THE INVENTION

Patients, particularly senior citizens and those requiring at least minimal physical assistance, are confronted with the ongoing need of taking each prescribed medication during a specified day and time, in a specified quantity, and without accidentally mixing a pill with other pills that can produce unwanted side effects. Mental lapses or confusion by the elderly often lead to taking an incorrect combination of pills.

Nonadherence by patients in the public domain to prescribed medications is steadily increasing, leading to thousands of deaths a year, especially for the elderly and those requiring at least minimal physical assistance. The extra cost of health care for patients made seriously or terminally ill due to nonadherence to prescribed medications is a huge burden on the public health system.

Many pill organizers and automated pill dispensers are known from the prior art; however, these prior art devices are patient-specific and for home use. Caregivers wishing to supply the patient with a correct combination of medications at any given time have to procure large quantities of medications in advance and to load the device. Often, an excessively high quantity of pills is purchased, and the excess pills that have not been loaded into the device are left in a storage area and are forgotten. The expiration date of many of these pills consequently passes, causing unnecessary wastage. Other existing solutions relate to automated dispensing cabinets (ADC), which is a computerized medicine cabinet for hospitals and healthcare settings. ADCs allow medications to be stored and dispensed near the point of care while controlling and tracking drug distribution. However, such solutions are not directed for a personal use and are lack the ability to provide comprehensive solution to an individual user regardless the location of the user.

It is an object of the present invention to provide a medication dispensing machine that will reliably dispense a correct combination of medications for each of a large number of patients at a physician-prescribed time.

It is an additional object of the present invention to provide a medication dispensing machine that minimizes medication wastage.

It is an additional object of the present invention to provide a system that ensures compliance with a medication regimen.

Other objects and advantages of the invention will become apparent as the description proceeds.

### SUMMARY OF THE INVENTION

A medication dispensing machine for a public domain comprises storage apparatus within which is stored a sufficient supply of each medication needed by all patients of the public domain during a predetermined time period; identification equipment by which each of a plurality of patients of the public domain is able to be authenticated; a controller for coordinating various machine operations; and dispensing apparatus cooperating with said controller and said storage apparatus which is configured to provide a given patient with a prescribed dosage for each of a prescribed combination of medications at a prescribed time following authentication.

The medication dispensing machine may further comprise one or more of the following:
A. communication equipment configured to communicate with, and retrieve information from, at least one computerized device that provides an updated list of patients who need to take medications on a regular basis as well as updated prescription and medical information for each patient, wherein the controller is configured to process the received information and to command the dispensing apparatus as to which combination of medications to dispense at the prescribed time;
B. a reliability module configured to interface with the controller and the dispensing apparatus and to assure that the medications to be dispensed conform to types, dosages, amounts and timing prescribed for the given patient;
C. a stock management system in data communication with the reliability module for maintaining an updated count of all medications held in the storage apparatus following a dispensing operation and for issuing a low stock signal when the updated count is less than a predetermined low threshold;
D. a compliance monitoring module configured to determine whether the given patient has taken each of the prescribed combination of medications within a predetermined duration following a dispensing time and to transmit a compliance indicating signal if the given patient has taken each of the prescribed combination of medications within the predetermined duration following the dispensing time; and
E. portioning apparatus for wrapping or packaging the prescribed combination of dispended medications, such as configured to individually wrap or package each portion of dispensed medications that is needed during an anticipated travel time and to specify when each portion is to be taken.

The at least one computerized device may be a remote server, such as a cloud based server, which is adapted to retrieve information from a public domain computer and from medical authority servers, or may be a public domain computer and one or more medical authority servers, the one or more medical authority servers supplying the updated prescription and medical information which is correlated with an identification number of a patient that is provided by the public domain computer.

According to an embodiment of the invention, the medication dispensing machine further comprises communication channel configured to communicate with a dedicated patient personal application.

According to an embodiment of the invention, the dispensing apparatus is configured for individually packaging each portion of medications that is needed to be taken at a different time and specifying when each portion is to be taken. According to an embodiment of the invention, each medication portion to be taken at a different time is preferably wrapped or packaged separately, and the corresponding prescribed time for the medication portion to be taken is labeled. This separate packaging may further be utilized to avoid contingencies between medicines

A medical data management system comprises a server; and a plurality of medication dispensing machines in data communication with said server, each of said medication dispensing machines being deployed in a different public domain and configured to dispense a prescribed combination of medications to each registered patient of said different public domain at a prescribed time following authentication and to transmit a compliance indicating signal to said server upon determining that the registered patient has taken each of the prescribed combination of medications within a predetermined duration following a dispensing time, wherein said server is operable to process data related to said transmitted compliance indicating signal and to associate said processed data with a known or forecasted medical event.

In one aspect, the medical data management system further comprises a plurality of electronically accessible nurse stations in data communication with the server, each of which is associated with a corresponding medication dispensing machine and is manned with medical personnel who are responsible for ensuring health care of all the patients registered at the corresponding medication dispensing machine. The server is operable to transmit an alert signal to one of the nurse stations upon identifying a medical event following processing the data related to the transmitted compliance indicating signal to indicate that one of the medical personnel is needed to provide caregiving services to a given patient associated with the alert signal.

Each of the nurse stations comprises stationary or portable communication apparatus, enabling data communication with the server by means of one or more devices selected for example from the group consisting of tablets, personal computers, smartphones, and alertable wearable devices. Each of these one or more devices is configured to receive from the server an updated list of patients by means of an application running on a processor thereof.

In one aspect, the medical data management system further comprises one or more physiological data sources, such as wearable devices worn by a patient, invasive/non-invasive medical equipment, 3rd party data such as blood test results, etc. For example, each of the wearable devices comprising one or more sensors that are configured to detect physiological data (e.g., data associated with bodily liquids, such as the bloodstream, physiological measurements, such as heart rate, etc.) of the patient that is indicative that a certain prescribed medication has been ingested, and a transmitter configured to transmit a compliance indicating signal containing the detected physiological data directly or indirectly to the server, wherein the server is configured to generate an alert signal if said compliance indicating signal has not been received within a predetermined duration following a prescribed time for ingesting the prescribed medication.

In one aspect, the server is cloud based.

In one aspect, the server is located at a same public domain as one of the medication dispensing machines, is remotely located relative to one of the medication dispensing machines, or is a centralized server.

A medication compliance monitoring system comprises a wearable device worn by a user, and a centralized monitoring station, wherein said wearable device comprises one or more sensors that are configured to detect physiological data associated with bodily liquid of the user that is indicative that a certain prescribed medication has been ingested, and a transmitter configured to transmit a compliance indicating signal containing the detected physiological data directly or indirectly to said monitoring station, wherein said monitoring station is configured to generate an alert signal if said compliance indicating signal has not been received within a predetermined duration following a prescribed time for ingesting the prescribed medication.

In another aspect, the present invention relates to a method of dispensing medication in a public domain, comprising:
a) providing one or more medication dispensing machines suitable to store a sufficient supply of each medication needed by patients of the public domain during a predetermined time period;
b) authenticating a patient upon receiving a request to provide said patient with a prescribed dosage for each of a prescribed combination of medications at a prescribed time; and
c) dispensing, via one of said medication dispensing machines, at least one prescribed dosage to be taken at a prescribed time.

According to an embodiment of the invention, the method further comprises communicating with, and retrieving information from, at least one computerized device that provides an updated list of patients who need to take medications on a regular basis as well as updated prescription and medical information for each patient, wherein the medication dispensing machine is configured to process the received information and to command a dispensing apparatus of said medication dispensing machine as to which combination of medications to dispense at the prescribed time or to dispense a plurality of prescribed dosages each to be taken at a different prescribed time.

According to an embodiment of the invention, the method further comprises assuring that the medications to be dispensed conform to types, dosages, amounts and timing prescribed for the patient.

According to an embodiment of the invention, the method further comprises maintaining an updated count of all medications held in each of the one or more medication dispensing machines following a dispensing operation and for issuing a low stock signal when the updated count is less than a predetermined low threshold.

According to an embodiment of the invention, the method further comprises determining whether the patient has taken each of the prescribed combination of medications within a predetermined duration following a dispensing time or following a prescribed time and transmitting a compliance indicating signal if the given patient has taken each of the prescribed combination of medications within the predetermined duration following the dispensing time or the prescribed time.

According to an embodiment of the invention, the method further comprises wrapping or packaging the prescribed combination of dispended medications, such as configured to individually wrap or package each portion of dispensed medications and specifying when each portion is to be taken.

According to an embodiment of the invention, the method further comprises enabling communication with medical personnel and/or non-medical practitioners, or enabling to obtain a pre-pharmacist approval for specific medications.

According to an embodiment of the invention, the method of dispensing medication in the public domain comprises:
a) receiving at least one prescription for a given patient;
b) receiving authorization for each received prescription in accordance with regulation;
c) upon authorization, directing the given patient to at least one available medication dispensing machine that is capable of providing the given patient with the needed prescribed medications;
d) identifying the given patient and accordingly allowing the medication dispensing machine to dispense at least one prescribed dosage to be taken at a prescribed time;
e) setting reminder for each prescribed dosage in accordance with its prescribed time;
f) receiving confirmation indicative of the consumption of each prescribed dosage; and
g) analyzing information relative to medication consumption by patients.

### BRIEF DESCRIPTION OF THE DRAWINGS

In the drawings:

FIG. 1 is a schematic illustration of a medical data management system, according to an embodiment;

FIG. 2 is a flow chart regarding operations involved in the dispensing of medication in conjunction with a public domain deployed machine, according to an embodiment;

FIG. 3A is a schematic illustration of a medication dispensing machine, according to an embodiment;

FIG. 3B schematically illustrates a portioning apparatus of the medication dispensing machine of FIG. 3A, according to an embodiment of the invention;

FIG. 3C schematically illustrates a sachet packaging of a portion of a prescribe medication, according to an embodiment of the invention;

FIG. 3D schematically illustrates a plurality of dispensed sachet packaging, according to an embodiment of the invention;

FIG. 4 is a schematic illustration of a bracelet shown in perspective view that is operable in conjunction with a medication compliance monitoring system, according to an embodiment; and

FIG. 5 schematically illustrates a medical data management system, according to another embodiment of the invention.

### DETAILED DESCRIPTION OF THE INVENTION

A medication dispensing machine deployed in a public domain is configured to dispense medication to a patient when needed. A typical public domain in which the medication dispensing machine is deployed is an assisted living facility, although other suitable public domains include a nursing home, a rehabilitation center, hospital a pharmacy, etc.

In one aspect, a prescribed dosage for each of a combination of medications is reliably dispensed to each of a plurality of patients at a prescribed time following patient authentication. The medication dispensing machine may operate in conjunction with a system for monitoring compliance with a medication regimen. The use of the medication dispensing machine significantly reduces the manpower usually needed in such public domains for ensuring patient compliance with a medication regimen and also minimizes medication wastage. In another aspect, a prescribed dosage for each of a combination of medications is reliably dispensed to each of a plurality of patients following patient authentication, wherein each prescribed dosage is adapted to be easily consumed in a prescribed time. For example, by being dispensed in a suitable packing or arrangement form suitable to be used in prescribed times.

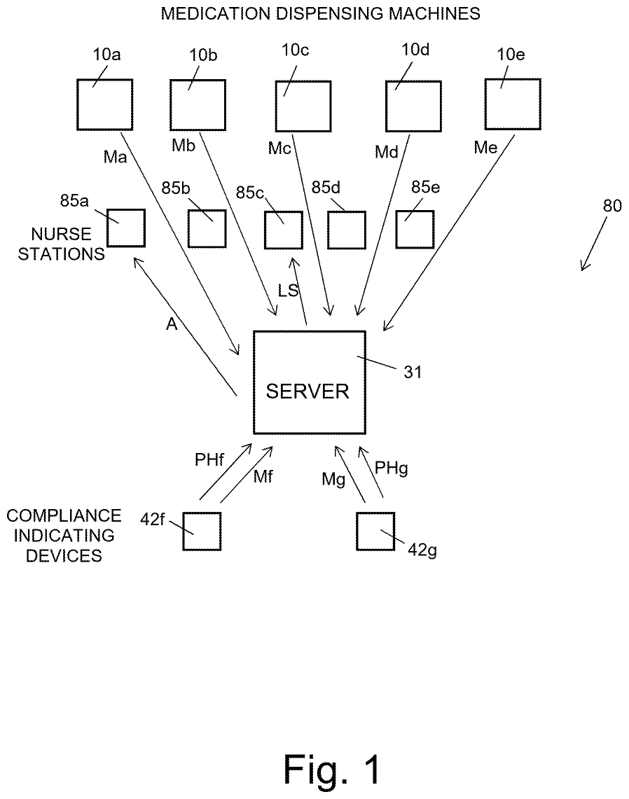

FIG. 1 schematically illustrates a medical data management system 80. In this embodiment, remote server 31, which may be a cloud server (e.g., a global cloud management server), is in data communication with a plurality of distributed medication dispensing machines, for example medication dispensing machines 10a-e, a plurality of distributed bracelets, for example bracelets 42f-g, or with other compliance indicating devices. The operation of a medication dispensing machine and of a compliance indicating bracelet will be described hereinafter.

Each of medication dispensing machines 10a-e is deployed in a different public domain and is configured to dispense a prescribed combination of medications to each registered patient of a given public domain at a prescribed time. Upon determining that a patient took the combination of medications at the prescribed time, the corresponding medication dispensing machine is configured to transmit one of the compliance indicating signals Ma-e to server 31, via any suitable communication means or channels, either directly to server 31 or indirectly, e.g., via a smartphone application.

When a patient is traveling, or is otherwise unable to physically access a medication dispensing machine, a dedicated application (e.g., a smartphone application) or a wearable device (e.g., a bracelet is worn) may be used. According to an embodiment of the invention, a wearable device in form of a bracelet is configured to detect physiological data of the patient and to directly or indirectly transmit the detected physiological data by one of the physiological data indicating signals PHf-g, which may be indicative of compliance with the medical regimen, to server 31. A bracelet, or other worn compliance indicating device, may also directly or indirectly transmit one of the compliance indicating signals Mf-g to server 31. In another embodiment, the dedicated application is configured to interact with the patient in accordance with communication with server 31 (e.g., generating reminders in accordance with dispensed prescribed combination of medications, receiving feedback from the patient, etc.) as will be described in further details hereinafter.

Remote server 31 is able to store patient-specific data that is acquired from each of the medication dispensing machines of from each of the compliance indicating devices with which it is in data communication, including the time when the medication was taken, degree of compliance, patient's body measurements or other physiological data at the time when the medication was taken, anomalous reactions, if any, to the medication, and local climatic conditions. Through the vast quantity of acquired data at the big data scale, server 31 is able to analyze the data to determine patient behavior and trends, cluster patients with similar behavior, and forecast future medical needs by associating the taking of medication with a medical event.

Electronically accessible medical personnel stations (MPS), such as nurse stations 85a-e, are in data communication with server 31. Each MPS may be manned with medical personnel (e.g., physician, nurse, etc.) who are responsible for ensuring the health care of all patients registered at the corresponding medication dispensing machines 10a-e, as well as the reliable operation of the corresponding medication dispensing machine 10a-e. A medical personnel station may be equipped with stationary communication apparatus in data communication with server 31, such as a control room to monitor and visualize the various patients in the public domain, a workstation to access real-time patient information, one or more personal computers, a call system to communicate with other medical professionals and with patients, and a real-time location system to identify the current location of medical equipment and patients associated with the public domain. An MPS may also be equipped with portable communication apparatus, such as a tablet, smartphone, and alertable wearable device, linked to one or more of the stationary communication apparatus, or directly linked to server 31.

When the communication apparatus of an MPS receives an alert signal A from server 31, after the acquired patient-specific data has been processed and a medical contingency has been identified, one of the medical personnel is needed to provide caregiving services to the patient associated with the alert signal, either locally or remotely (e.g., via a mobile device—voice or application), and possibly to contact a designated family member or friend. Alternatively, a physician who is one of the medical personnel may have to change a prescription or a medication regimen for one of the registered patients. When the communication apparatus of an MPS receives a low stock LS signal from server 31, which may also be indicative of a medication related expiration date that has passed, one of the medical personnel is needed to replenish the supply of the medication within the corresponding medication dispensing machine which is associated with the low stock signal. When equipped with a processor, the communication apparatus of an MPS may receive an updated list of patients from server 31 by means of an application running on its processor.

While the MPS is referred to as a nurse station, it will be appreciated that the station may be manned not only by nurses and other medical practitioners such as physicians, but also by non-medical practitioners such as pharmacists, a stock administrator, service center and helpdesk, administrative or IT personnel working at a back-office station, and technical assistants working at a help desk station. These people will receive an alert individually or collectively. Alternatively, an MPS may be unmanned, and may autonomously process a received alert signal in order to provide caregiving services to the patient associated with the alert signal.

It will be appreciated that more than one MPS may be associated with a given public domain.

Medical data management system 80 may also comprise other Internet-of-Thing (IoT) devices or other devices functioning as a sensor for detecting abnormal patient behavior. The system may for example comprise a machine learning module that relies on these IoT devices as sources for the input to the machine learning module.

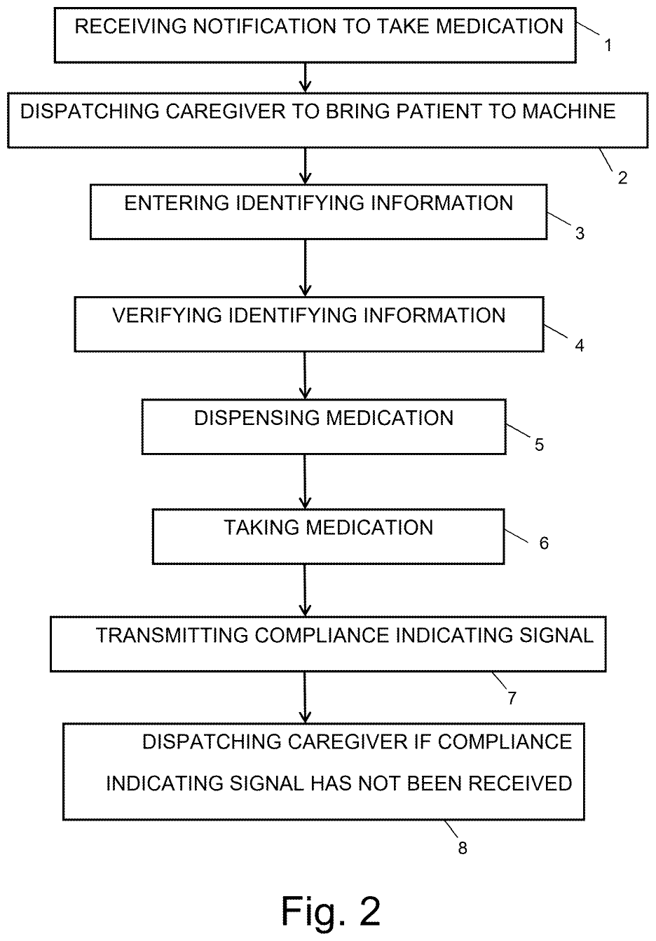

Broadly speaking, FIG. 2 illustrates a flow chart indicating the basic operations involved in the dispensing of medication to a patient in conjunction with a public domain deployed machine, according to an embodiment of the invention. If the patient is known to be located outside of the boundaries of the public domain, one or more other operations are carried out, as will be described hereinafter.

In step 1, a patient receives a notification that medication should presently be taken. As referred to herein, a medication is “taken” by being ingested for example when the medication is in pill form, inhaled for example when the medication is a bronchodilator, injected for example when the medication such as insulin is self-administered, or topically applied for example when the medication is an ointment, so that the patient's body will benefit from the curative substance included within the medication. The notification could be transmitted as a text message, such as an application derived message or an SMS message, to the patient's mobile device or to the mobile device of a specified family member, or as an e-mail message. Alternatively, or in combination, the notification can be a verbal announcement made over a loudspeaker of the public domain. After the patient is made known of the notification, the patient approaches the medication dispensing machine and enters identifying information in step 3. If a predetermined time has elapsed following generation of the notification, a caregiver is dispatched, for example by the medical personnel station through the intervention of the remote server or by the machine, in step 2, generally by means of an electronically generated message, to personally bring the patient to the machine.

After the patient's identifying information is verified in step 4, the prescribed medication or medications are dispensed in step 5. After the prescribed medication has been taken in step 6, a compliance indicating signal is transmitted, for example to a centralized monitoring station or to a controller of the medication dispensing machine, in step 7. The patient is reminded to take the prescribed medication in step 8, generally by means of an electronically generated message, for example which is intended to dispatch a caregiver to the patient, if the compliance indicating signal has not been received within a predetermined time following dispensing of the medication.

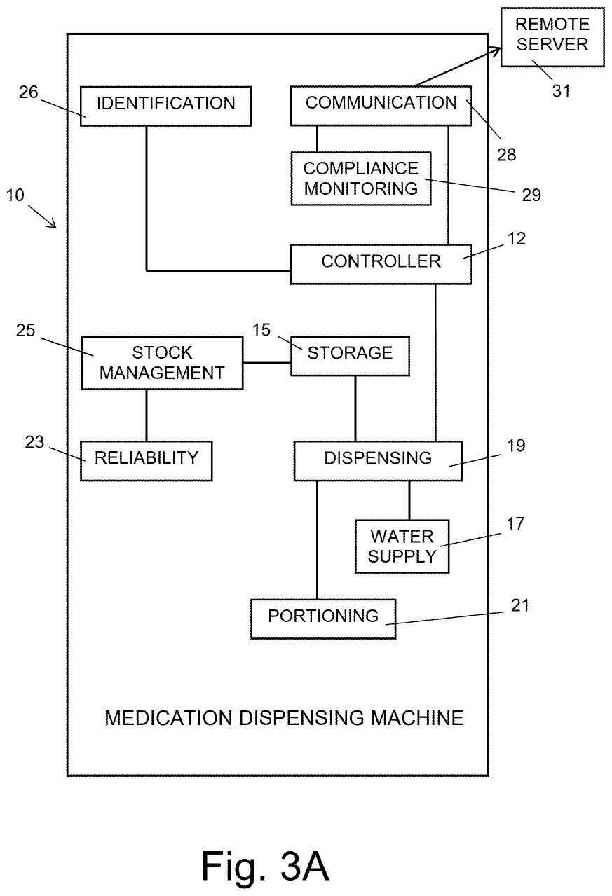

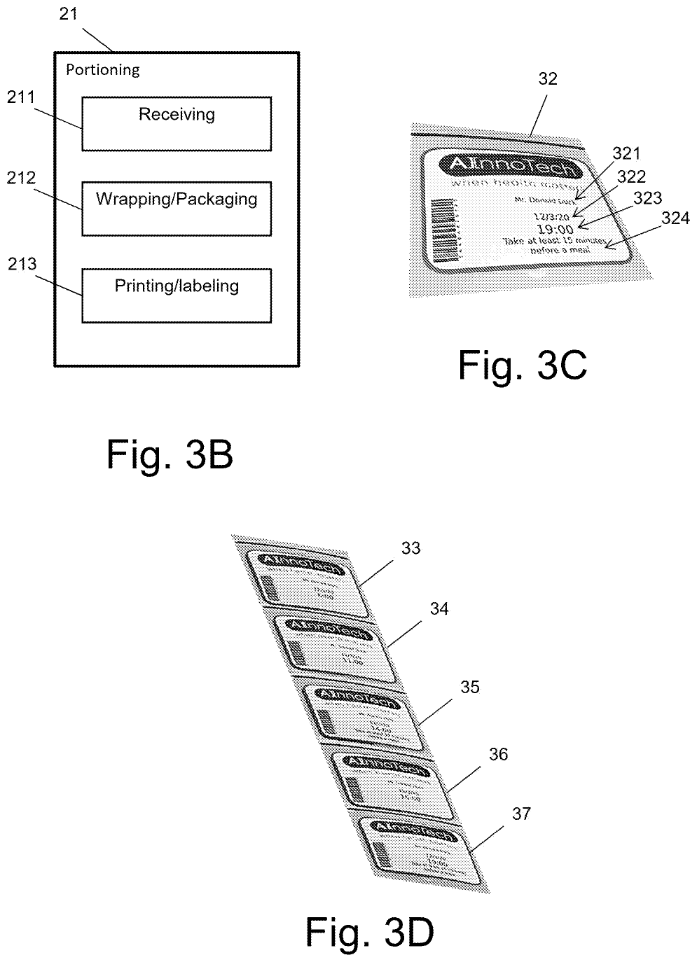

FIG. 3A schematically illustrates a medication dispensing machine 10 according to an embodiment of the invention.

Medication dispensing machine 10 comprises a controller 12 for coordinating various machine operations, and medication storage apparatus 15, for example a plurality of individualized cartridges, within each of which a sufficient supply of a given medication needed by all patients of the public domain during a predetermined time period, e.g. a month, is stored. Each cartridge may be reusable in order to replenish the medication supply, or alternatively may be disposable. A water supply connection 17 is also provided, whether from a water supply network or from a water tank or reservoir. Dispensing apparatus 19 is in communication with medication storage apparatus 15 and with water supply connection 17 in order to provide a given patient with a prescribed combination of medications. Some medications may be dispensed in pill form. Some medications may be liquid and may have to be diluted with water provided via connection 17. Water may also be discharged via connection 17 to assist the patient in swallowing the dispensed pills. Portioning apparatus 21 may receive the medications discharged by dispensing apparatus 19 and arrange them in a user friendly fashion, such as within a small cup or on a platter, or within an easy to remove wrapping or packaging material, such as a blister pack, a bottle or a sachet packaging 32 as schematically shown in FIG. 3C. Each different type of medication may be wrapped or packaged separately. If requested by the patient, such as if planning to travel, portioning apparatus 21 may individually package each portion of medications that is needed during the anticipated travel time and specify when each portion is to be taken. Each medication portion to be taken at a different time is preferably wrapped or packaged separately, and the corresponding prescribed time for the medication portion to be taken is preferably clearly labeled or printed on each package (e.g., by using printing and/labeling means that can be part of the portioning apparatus 21). For example, FIG. 3B schematically illustrates portioning apparatus 21 according to an embodiment of the invention. In this example, portioning apparatus 21 comprises a receiving unit 211 for receiving medications discharged by dispensing apparatus 19, a packaging/wrapping unit 212 packaging one or more prescribed dosages (e.g., each individual prescribed dosage can be wrapped or packed separately and may contain combination of medications), and a printing/labeling means for clearly indicating on each package the corresponding prescribed time for the medication portion to be taken. FIG. 3C schematically illustrates an example of a packed dosage in form of a sachet packaging 32 on which the corresponding prescribed time for the medication portion to be taken as well as other relevant information are labeled, such as patient's name 321, date 322, prescribed time 323 and optional instruction 324 associated with the usage of the medication (e.g., “take at least 15 minutes before a meal”).

Medication dispensing machine 10 is an automated drug distribution machine that enables to provide automated, customized, accessible, available and accurate medicines distribution divided to doses by time according to updated relevant medical data. FIG. 3D schematically illustrates an example of a plurality of sachet packaging 33-37 that are dispensed in a roll-like form by medication dispensing machine 10. Each sachet is labeled with its own prescribed time and may contain a combination of medications.

Dispensing apparatus 19 operates in conjunction with a reliability module 23 which assures that the dispensed medications conform to the types, dosages, amounts and timing prescribed for the given patient. Reliability module 23 in turn interfaces with a stock management system 25 for maintaining an updated count of all medications held in medication storage apparatus 15 following a dispensing operation, including an identification of the batch and brand name of the medication and an updated volume of liquid, powder and ointment medications after having been dispensed from a local repository. Stock management system 23 issues a low stock signal when the updated count approaches a predetermined low threshold. The low stock signal may be transmitted directly to the remote server, or, alternatively, may be transmitted to the corresponding medical personnel station. By being able to identify the batch of a stocked medication, those medications can be easily removed from medication storage apparatus 15 if found to have been recalled, for example if adverse health effects are liable to occur.

According to an embodiment of the invention, medication dispensing machine 10 may comprise one or more additional modules/units such as audit, transactions means, etc. (not shown).

In order to verify that the correct patient is to receive the prescribed medication, the medication dispensing machine comprises identification equipment 26 including, but not limited to, an application verification, an RF reader operating for example in conjunction with a proximity sensor to read a patient-carried transmitter such as a tag, a camera for identifying the patient's facial characteristics, and a biometric system for recognizing one or more patient physiological characteristics such as fingerprint, palm veins, DNA, palm print, hand geometry, iris recognition, and retina characteristics, which have been previously entered into controller 12 during an enrollment process.

Communication equipment 28 communicates with, and retrieves information from, various servers and computers with which it is authorized to communicate, including a public domain computer that provides an updated list of patients who need to take medications on a regular basis as well as their identification number, and medical authority servers for supplying updated prescriptions and medical information for each patient, as well as a dedicated patient personal application (e.g., a smartphone application) as will be described in further details hereinafter. Alternatively, communication equipment 28 may communicate with remote server 31, which may be cloud-based and adapted to retrieve information from the public domain computer and from medical authority servers. Access to the medical information ensures that correct prescribed medications will be dispensed to the patient following verification of the entered identifying information.

According to an embodiment of the invention, a dedicated patient personal application (e.g., that runs on a patient mobile device) can be used in accordance with server 31 as a personal assistance for medicine distribution and consumption. The application is synchronized with server 31 and with prescribed dispensed dosages provided by medication dispensing machine 10 and may remind the patient to timely consume the medications as well as configured to receive feedback from the patient before/after the consumption (e.g., physical measurements/readings, voice analysis, questionnaires, etc.). For example, the application may remind the patient to consume the medication by showing a visual representation of a corresponding sachet packaging for the current medication portion to be taken. Thus, the patient may easily identify the correct sachet packaging and thereby may avoid consumption errors. According to an embodiment of the invention, the application can be used to enable communication like chat, voice or videoconference with medical personnel such as physician, nurse or enable to obtain a pre-pharmacist approval for specific medications (in accordance with regulations).

Many jurisdictions require a pharmacist, or a physician, to provide oral consultation to a patient whenever a prescribed medication has not previously been dispensed to the patient, or whenever the prescribed medication has not been dispensed to the patient in the same dosage, form, strength or with the same written directions.

Medication dispensing machine 10 may comprise a video player, or any other type of media player, for displaying or emitting a live or taped pharmacist-conducted oral or videoconference consultation. The media player may be in data communication with controller 12 and reliability module 23. In the pharmacist-conducted oral consultation, directions for taking the prescribed medication are generally described, as well as common side effects that may be encountered and the importance of compliance with the directions.

A compliance monitoring system 29 identifies when the patient complies with the medication regimen set by the patient's physician. Compliance monitoring system 29 may comprise a camera for capturing images of the patient after receiving the dispensed medications, and an image processor for analyzing the captured images to determine whether they are indicative that all of the dispensed medications have been taken at the prescribed time.

Alternatively, system 29 comprises a patient worn device that is able to detect physiological changes in the content of the patient's blood or other bodily fluid, in response to ingestion of the medication. The patient worn device in turn transmits the acquired patient physiological data to communication equipment 28 and then to controller 12. The transmitted data may be subsequently transmitted to remote server 31 for analysis and documentation of the patient related data.

Alternatively, system 29 interfaces with the dedicated application running on a patient's mobile device. This application is adapted to interface with signals transmitted from an ingestible sensor embedded in a pill dispensed to the patient indicating that the pill has been ingested. With this capability, system 29 constitutes a digital ingestion tracking system. The transmitted data may be subsequently transmitted to remote server 31.

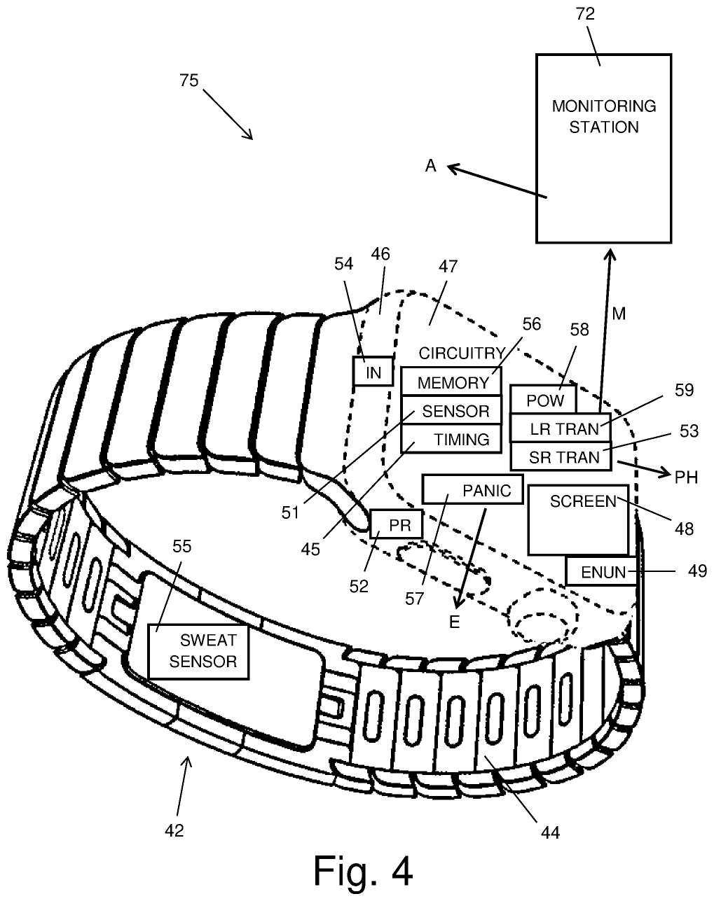

FIG. 4 schematically illustrates a bracelet 42 that is operable in conjunction with a compliance monitoring system 75. Other similar functioning wearable devices may also be suitable.

Bracelet 42 comprises an elongated strap 44, or other type of band, worn on the wrist of a patient and a casing 46 secured to strap 44 within which is housed circuitry 47 adapted to perform medication compliance monitoring operations. Circuitry 47 may comprise one or more sensors 51 that are configured to detect physiological data associated with bodily liquids of the user, such as the bloodstream or secretions, e.g. urine and saliva, which is indicative that a certain prescribed medication has been ingested, one or more sweat sensors 55 that are configured to detect sweat related physiological data by measuring various biomarkers, such as pH, lactate, chloride and glucose levels and the patient's body temperature, to indicate that a certain prescribed medication has been ingested in lieu of, or in addition to, the one or more sensors 51, a short-range transmitter 53, e.g. a Bluetooth transmitter, configured to transmit a signal PH containing the detected physiological data to a mobile device of the patient with which it is paired for subsequent transmission to the controller of the medication dispensing machine and/or to server 31, a memory device 56 for temporarily storing the detected physiological data if the mobile device is not operational for any reason, and a power source 58 such as a battery for powering circuitry 47. Circuitry 47 may also comprise timing circuitry 45 for powering circuitry 47 only during predetermined periods of time that may coincide with times when medication should be taken.

Alternatively, circuitry 47 may be equipped with a long-range transmitter 59 that has cellular functionality, so that a signal PH containing the detected physiological data may be transmitted directly to the controller of the medication dispensing machine and/or to server 31.

Bracelet 42 has particular utility when the patient is located remotely to the medication dispensing machine such as when visiting relatives, or is bedridden and is unable to access the machine.

A flexible screen 48 in communication with circuitry 47 may be provided with bracelet 42 in order to notify the patient by a visual message when the medications should be taken. An enunciator 49 may also be provided to issue an audible notification. One or more user-friendly input devices 54 facilitate a change in settings, such as a change in time zone. When bracelet 42 has cellular functionality, the patient may be urgently reminded by the system that the medications have not yet been taken. Also, circuitry 47 may comprise a processor 52 adapted to compare the detected physiological data with physiological data stored within memory device 56. If the detected physiological data is found to be similar to the stored physiological data in accordance with stored instructions, processor 52 will generate a compliance indicating signal M. Processor 52 will then command long-range transmitter 59 to transmit compliance indicating signal M via the cellular network to the controller of the medication dispensing machine and/or server 31, or will command short-range transmitter 53 to transmit compliance indicating signal M via the cellular network to the patient's mobile device and then to the controller of the medication dispensing machine.

Bracelet 42 may also comprise a media player for playing a pharmacist-conducted oral consultation, which may be displayed on screen 48 and emitted by enunciator 49.

It will be appreciated that bracelet 42 is suitable for transmitting a compliance indicating signal M to other types of centralized monitoring stations 72, in addition to the medication dispensing machine. Monitoring station 72 may be configured to generate an alert signal A if compliance indicating signal M has not been received within a predetermined duration following a prescribed time for ingesting the prescribed medication.

Bracelet 42 may also be equipped with a panic button 57 in data communication with processor 52. Panic button 57 may comprise a motion sensor which is calibrated to generate an emergency signal E, to be transmitted via short-range transmitter 53 or long-range transmitter 59 to monitoring station 72, e.g., if the patient does not move for more than a predetermined time or if it senses that the patient fell. Panic button 57 may also be pressed by the patient or by a caregiver. Optionally, bracelet 42 may comprise a microphone element that can be used to indicate when a patient is in emergency and calling for support.

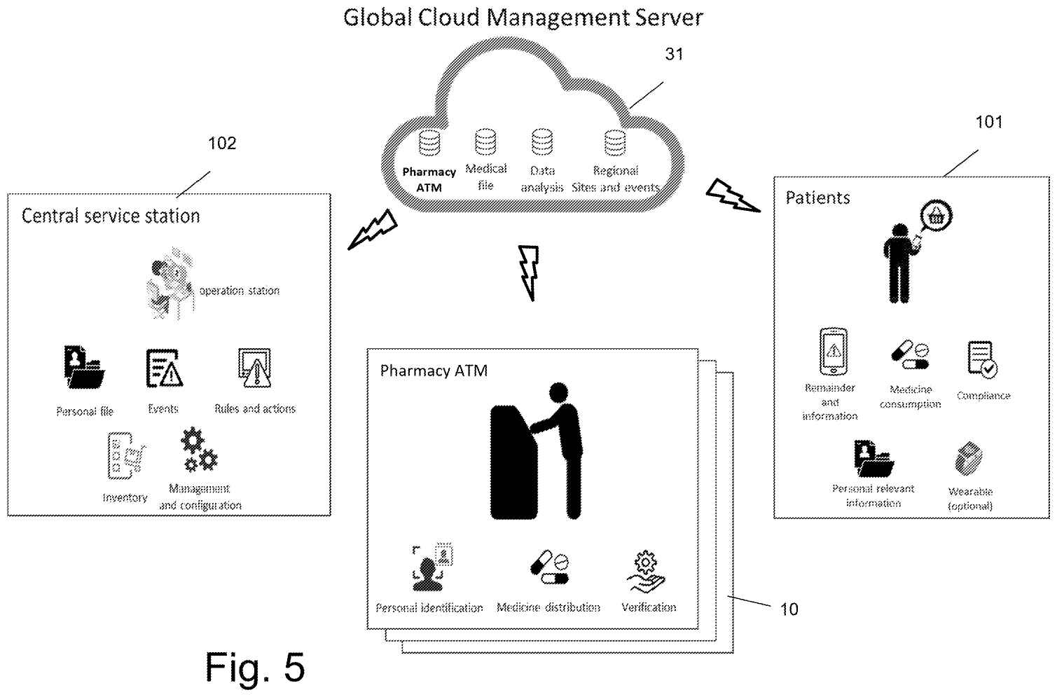

FIG. 5 schematically illustrates a medical data management system 90, according to an embodiment of the invention. In this embodiment, medication dispensing machine 10 functions as a pharmacy Automatic Teller Machine (ATM) that is available 24/7 and is highly accessible, while server 31 functions as a global cloud management server that is configured to manage a plurality of such pharmacy ATMs. Server 31 may involve the following tasks and procedures: manage medical files of patients, perform data analysis (medication consumption data, data in response to ingestion of the medication, gender, age, medical condition, diet etc., that for example, can be used to provide forecasting and trends, medicine insights according to patients' classification, etc.), and manages regional sites and events. The pharmacy ATM may involve personal identification of a patient/user, medicine distribution (e.g., in form of sachets packaging 32-37 as schematically indicated in FIGS. 3C and 3D) and consumption verification.

According to an embodiment of the invention, system 90 comprises at least one central service station 102 that is configured to function as a service center and helpdesk and a dedicated patient personal application 101. Central service station 102 may involve the following: managing users' personal files, managing events, setting system's rules and actions, enabling management and configuration of the system, providing remote assistance—medical, pharmacist and technical, monitoring pharmacy ATMs and patient's vitals, managing inventory (e.g., of each pharmacy ATM). Application 101 may run on a patient mobile device (e.g., smartphone) and it may comprise medicine consumption reminders/alerts or other indications, compliance, personal information, and it also may interface with a wearable device (e.g., as described with respect to FIG. 4 hereinabove).

According to an embodiment of the invention, dedicated patient personal application 101 functions as a personal assistance for medicine distribution and consumption, and may involve one or more of the followings tasks:

Providing medicine prescriptions;

Allocating pharmacy ATM based on the patient's prescriptions and location;

Generating medicine consumption reminders and enabling compliance;

Storing personal relevant information—location, Payment, family, deficiencies, contacts etc.;

Providing medicine information and consumption instructions;

Enabling online and offline functionality;

Interfacing with wearable devices—optional; and

Enabling personal physician or pharmacist consultancy—optional.

According to an embodiment of the invention, server 31 functions as a cloud based global management layer, and may involve one or more of the followings tasks:

Managing personal medical files (relevant information—integration with relevant systems (e.g., 3^{rd} party medical related sources, insurance companies, etc.);

Managing pharmacy ATM—locations, inventory, consumptions, malfunctions etc.;

Processing personal information—activity information, classification, compliance, contacts, etc.;

Enabling data management (audit, data, video, audio, etc.);

Processing data for providing analytic, trends and forecasting;

Setting rules and events;

Providing medical and service assistance;

Allocating medicine; and

Analyzing data for providing contingencies between medicines.

According to an embodiment of the invention, the system can be configured to enable special services, such as configuring the system to operate in a hospital environment (e.g., where medical stuff may assist/serve the patients), providing dedicated pharmacy ATM or medication dispensing machine 10 configured to provide cannabis, narcotics or sensitive medications, etc.

All the above will be better understood through the following illustrative and non-limitative examples.

According to an embodiment of the invention, a method of dispensing medication in a public domain may involve the following steps:
receiving at least one prescription for a given patient;
receiving authorization for each received prescription in accordance with regulation, e.g., as set forth by health organizations, Ministry of Health, etc.;
upon authorization, directing the given patient to at least one available pharmacy ATM that is currently suitable to provide the patient with the needed prescribed medications. For example, based on the current location of the patient, the dedicated application that runs on the patient's mobile device, may show the patient one or more nearby pharmacy ATMs that are suitable to provide the required prescribed medications;
after arriving at the relevant pharmacy ATM, identifying the given patient and accordingly dispensing at least one prescribed dosage to be taken at a prescribed time;
setting reminder(s) for each prescribed dosage in accordance with its prescribed time, e.g., the dedicated application may remind the patient (or may send alerts/reminders) to timely consume each prescribed dosage of medication;
receiving confirmation indicative of the consumption of each prescribed dosage; and
Analyzing information relative to medication consumption and patients to identify trends, efficiency of treatment (e.g., according to different sections and population, age, gender, etc.), inventory management, operational efficiency of the system, etc.

As will be appreciated by the skilled person the arrangements described in the figures results in a comprehensive solution for automatic medicine distribution in the public domain from prescription until consumption and beyond to the right person, divided to doses by time according to updated relevant medical data and able to provide valuable feedback.

While some embodiments of the invention have been described by way of illustration, it will be apparent that the invention can be carried out with many modifications, variations and adaptations, and with the use of numerous equivalents or alternative solutions that are within the scope of persons skilled in the art, without exceeding the scope of the claims.

1. A medication dispensing machine for a public domain, comprising:
a) storage apparatus within which is stored a sufficient supply of each medication needed by all patients of the public domain during a predetermined time period;
b) identification equipment by which each of a plurality of patients of the public domain is able to be authenticated;
c) a controller for coordinating various machine operations;
d) dispensing apparatus cooperating with said controller and said storage apparatus which is configured to provide a given patient with a prescribed dosage for each of a prescribed combination of medications at a prescribed time following authentication; and
e) communication equipment configured to communicate with, and retrieve information from, at least one computerized device that provides an updated list of patients who need to take medications on a regular basis as well as updated prescription and medical information for each patient, wherein the controller is configured to process the received information and to command the dispensing apparatus as to which combination of medications to dispense at the prescribed time.

2. (canceled)

3. The medication dispensing machine according to claim 1, wherein the at least one computerized device is a remote/cloud server which is adapted to retrieve information from a public domain computer and from medical authority servers, wherein the one or more medical authority servers supplying the updated prescription and medical information which is correlated with an identification number of a patient that is provided by the public domain computer.

4. (canceled)

5. The medication dispensing machine according to claim 1, further comprising: a) a reliability module configured to interface with the controller and the dispensing apparatus and to assure that the medications to be dispensed conform to types, dosages, amounts and timing prescribed for the given patient for each dose; and b) a stock management system in data communication with the reliability module for maintaining an updated count of all medications held in the storage apparatus following a dispensing operation and for issuing a low stock signal when the updated count is less than a predetermined low threshold.

6. (canceled)

7. The medication dispensing machine according to claim 1, further comprising a compliance monitoring module configured to determine whether the given patient has taken each of the prescribed combination of medications within a predetermined duration following a dispensing time, to transmit a compliance indicating signal if the given patient has taken each of the prescribed combination of medications within the predetermined duration following the dispensing time, and setting a reminder for each prescribed dosage in accordance with its prescribed time.

8. The medication dispensing machine according to claim 1, further comprising portioning apparatus for wrapping or packaging the prescribed combination of dispended medications, wherein the portioning apparatus is configured to individually wrap or package each portion of dispensed medications that is needed during an anticipated travel time and to specify when each portion is to be taken.

9. (canceled)

10. The medication dispensing machine according to claim 1, further comprising communication channel configured to communicate with a dedicated user application.

11. The medication dispensing machine according to claim 1, wherein the dispensing apparatus is configured for individually package each portion of medications that is needed to be taken at a different time and specifying when each portion is to be taken, wherein each medication portion to be taken at a different time is preferably wrapped or packaged separately, and the corresponding prescribed time for the medication portion to be taken is labeled.

12. A medical data management system, comprising:
a) a server; and
b) a plurality of medication dispensing machines in data communication with said server, each of said medication dispensing machines being deployed in a different public domain and configured to dispense a prescribed combination of medications to each registered patient of said different public domain at a prescribed time following authentication and to transmit a compliance indicating signal to said server upon determining that the registered patient has taken each of the prescribed combination of medications within a predetermined duration following a dispensing time,
wherein said server is operable to process data related to said transmitted compliance indicating signal and to associate said processed data with a known or forecasted medical event.

13. The system according to claim 8, further comprising a plurality of electronically accessible medical personnel stations in data communication with the server, each of which is associated with a corresponding medication dispensing machine and is manned with medical personnel who are responsible for ensuring health care of all the patients registered at the corresponding medication dispensing machine, wherein each of the medical personnel stations comprises stationary or portable communication apparatus.

14. (canceled)

15. The system according to claim 9, wherein each of the medical personnel stations is in data communication with the server by means of one or more devices selected from the group consisting of tablets, personal computers, smartphones, and alertable wearable devices, wherein each of the one or more devices is configured to receive from the server an updated list of patients by means of an application running on a processor thereof.

16. (canceled)

17. The system according to claim 9, wherein the server is operable to transmit an alert signal to one or more of the medical personnel stations upon identifying a medical event following processing the data related to the transmitted compliance indicating signal to indicate that one of the medical personnel is needed to provide caregiving services to a given patient associated with the alert signal, wherein the server is operable to dispatch the one of the medical personnel/caregiver/family member to the given patient for assistance if the compliance indicating signal has not been received within the predetermined duration following the dispensing time.

18. (canceled)

19. The system according to claim 8, further comprising one or more wearable devices worn by a patient, each of said wearable devices comprising one or more sensors that are configured to detect physiological data associated with bodily liquids of the patient that is indicative that a certain prescribed medication has been ingested, and a transmitter configured to transmit a compliance indicating signal containing the detected physiological data directly or indirectly to the server, wherein the server is configured to generate an alert signal if said compliance indicating signal has not been received within a predetermined duration following a prescribed time for ingesting the prescribed medication.

20. (canceled)

21. The system according to claim 12, further comprising a dedicated user application adapted to run from a mobile device and configured to communicate with the server and/or with medication dispensing machines.

22. The system according to claim 8, further comprising:
a) a wearable device worn by a user; and
b) a centralized monitoring station,
wherein said wearable device comprises:
i. one or more sensors that are configured to detect physiological data associated with bodily liquid of the user that is indicative that a certain prescribed medication has been ingested; and
ii. a transmitter configured to transmit a compliance indicating signal containing the detected physiological data directly or indirectly to said monitoring station,
wherein said monitoring station is configured to generate an alert signal if said compliance indicating signal has not been received within a predetermined duration following a prescribed time for ingesting the prescribed medication.

23. A method of dispensing medication in a public domain, comprising:
a) providing one or more medication dispensing machines suitable to store a sufficient supply of each medication needed by patients of the public domain during a predetermined time period;
b) authenticating a patient upon receiving a request to provide said patient with a prescribed dosage for each of a prescribed combination of medications at a prescribed time;
c) dispensing, via one of said medication dispensing machines, at least one prescribed dosage to be taken at a prescribed time; and
d) communicating with, and retrieving information from, at least one computerized device that provides an updated list of patients who need to take medications on a regular basis as well as updated prescription and medical information for each patient, wherein the medication dispensing machine is configured to process the received information and to command a dispensing apparatus of said medication dispensing machine as to which combination of medications to dispense at the prescribed time or to dispense a plurality of prescribed dosages each to be taken at a different prescribed time.

24. (canceled)

25. A method according to claim 15, further comprising assuring that the medications to be dispensed conform to types, dosages, amounts and timing prescribed for the patient, and maintaining an updated count of all medications held in each of the one or more medication dispensing machines following a dispensing operation and for issuing a low stock signal when the updated count is less than a predetermined low threshold.

26. (canceled)

27. A method according to claim 15, further comprising determining whether the patient has taken each of the prescribed combination of medications within a predetermined duration following a dispensing time or following a prescribed time and transmitting a compliance indicating signal if the given patient has taken each of the prescribed combination of medications within the predetermined duration following the dispensing time or the prescribed time.

28. A method according to claim 15, further comprising wrapping or packaging the prescribed combination of dispended medications, such as configured to individually wrap or package each portion of dispensed medications and specifying when each portion is to be taken.

29. A method according to claim 15, further comprising enabling communication with medical personnel and/or non-medical practitioners, or enabling to obtain a pre-pharmacist approval for specific medications.

30. (canceled)

31. A method according to claim 15, wherein dispensing medication in the public domain comprising:
a) receiving at least one prescription for a given patient;
b) receiving authorization for each received prescription in accordance with regulation;
c) upon authorization, directing the given patient to at least one available medication dispensing machine that is capable of providing the given patient with the needed prescribed medications;
d) identifying the given patient and accordingly allowing the medication dispensing machine to dispense at least one prescribed dosage to be taken at a prescribed time;
e) setting reminder for each prescribed dosage in accordance with its prescribed time;
f) receiving confirmation indicative of the consumption of each prescribed dosage; and
g) analyzing information relative to medication consumption by patients.
